(19) United States
(12) Patent Application Publication
(10) Pub. No.: US 2022/0034724 A1
(43) Pub. Date: Feb. 3, 2022

(54) CONCRETE SENSOR DEVICE AND SYSTEM

(71) Applicant: CONCRETE DATA SENSORS PTY LTD., Brisbane (AU)

(72) Inventors: Andrew CATHCART, Brisbane (AU); Adrian MAHER, Brisbane (AU); Anthony CHISHOLM, Brisbane (AU)

(73) Assignee: CONCRETE DATA SENSORS PTY LTD.

(21) Appl. No.: 17/502,369

(22) Filed: Oct. 15, 2021

Related U.S. Application Data
(63) Continuation-in-part of application No. PCT/AU20/50362, filed on Apr. 14, 2020.

(51) Int. Cl. G01K 1/024; G01N 33/38; G01K 1/08; G01P 1/02; G01P 1/00; G01D 21/02

(52) U.S. Cl. CPC G01K 1/024; G01N 33/383; G01D 21/02; G01P 1/023; G01P 1/06; G01K 1/08

(57) ABSTRACT

A structure sensor device monitors physical properties within a body of concrete, rock, soil or other structure. The structure sensor device can be embedded inside the body of the structure in use. The structure sensor device includes: a sealed housing; sensors for measuring physical properties within the body of structure, such as a temperature sensor and a moisture sensor; and a wireless transceiver for wirelessly communicating with a gateway device located outside of the body of structure. The wireless transceiver is configured for wirelessly communicating at frequencies of less than 1 GHz. There is a controller configured to obtain sensor data from the sensors, generate monitoring data using at least some of the sensor data, and cause the monitoring data to be transmitted to the gateway device using the wireless transceiver, at predetermined intervals.

### CROSS-REFERENCE TO RELATED APPLICATIONS

The present application claims priority under 35 U.S.C. Section 120 from U.S. Patent Application Ser. No. PCT/AU20/50362, filed on 14 Apr. 2020, entitled “CONCRETE SENSOR DEVICE AND SYSTEM”. See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to a structure sensor device for use in monitoring physical properties of a body of structure, such as concrete, ground, rock, soil or earth. and a system for monitoring physical properties of one or more bodies of structure using a plurality of the structure sensor devices. This application is a Continuation In Part (CIP) of PCT/AU2020/050362 (WO/2020/210861), the disclosure of which is fully incorporated herein by reference. The description before mostly relates to application to a concrete structure; however, the structure sensor device can also be embedded in ground, rock, soil, or earth structures for monitoring temperature, humidity, tilt, movement and vibration of earth and rock structures.

### 2. Description of Related Art Including Information Disclosed Under 37 CFR 1.97 and 37 CFR 1.98.

When constructing buildings or other structures involving bodies of concrete, it can be of critical importance to ensure that the concrete has cured to a level of maturity that will provide sufficient strength for safety before progressing to subsequent construction stages.

Due to the variability of concrete curing process subject to external environmental factors, traditional methods of predicting the strength of concrete are typically over conservative and can result in unduly long waiting times.

Concrete sensors for measuring the temperature and/or moisture levels of concrete can be installed in bodies of concrete to allow more direct monitoring of the concrete curing process and allow for more timely progression to subsequent stages of construction projects. However, existing concrete sensor systems suffer from a range of drawbacks.

For examples, many conventional solutions require wiring between concrete sensors that are embedded in the concrete and wireless transmitters or physical connection points located external to the concrete. Whilst some solutions have removed this requirement by providing wireless transmitters in the embedded concrete sensors, their transmission range is often very limited, such that a user will need to be in close proximity to the concrete sensor in order to obtain the sensor data using a smartphone or other reader device. Some solutions provide additional repeaters and hubs for collecting the sensor data and transmitting it to servers for processing without requiring user proximity, but at the expense of significantly increased system complexity and hardware requirements.

Furthermore, existing concrete sensor systems will typically be limited to only measuring temperature and/or moisture levels of concrete during the concrete curing process, and provide no further value once sufficient maturity has been reached.

It would be desirable to provide an improved concrete sensor device, and a system using such devices, which remove one of more of the above drawbacks of existing systems, or at least provide a useful alternative to existing systems.

WO2017031526A1 discloses a system for reporting the maturity of a concrete, including at least one temperature sensor for sensing and recording temperatures of the concrete over time, a data retrieving device for retrieving the recorded temperatures from the at least one temperature sensor, a transmitter for transmitting the recorded temperatures and corresponding reference information, a first server for receiving the recorded temperatures and the corresponding reference information, a second server for verifying the corresponding reference information with record data, and a processor, wherein, upon a positive verification of the second server, analyses the recorded temperatures, calculates the maturity of the concrete, and provides a report online to a selected person.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavor to which this specification relates.

### BRIEF SUMMARY OF THE INVENTION

In one broad form, an aspect of the present invention seeks to provide a structure sensor device for use in monitoring physical properties within a body of concrete, rock or soil structure, the structure sensor device being configured to be embedded inside the body of structure in use, the structure sensor device including: a sealed housing; sensors for measuring physical properties within the body of structure, including a temperature sensor and a moisture sensor; a wireless transceiver for wirelessly communicating with a gateway device located outside of the body of structure, the wireless transceiver being configured for wirelessly communicating at frequencies of less than 1 GHz; and a controller configured to obtain sensor data from the sensors, generate monitoring data using at least some of the sensor data, and cause the monitoring data to be transmitted to the gateway device using the wireless transceiver, at predetermined intervals.

In one embodiment, the sensors further include an accelerometer.

In one embodiment, the controller is configured to use sensor data obtained from the accelerometer to determine at least one of: a tilt angle of the structure sensor device; stiffness of the structure; and vibration in the structure.

In one embodiment, the structure sensor device is configured to operate under different operating modes, the controller being configured to: generate the monitoring data in accordance with a current operating mode; and cause the monitoring data to be transmitted to the gateway device at intervals selected in accordance with the current operating mode.

In one embodiment, the structure is concrete, and the operating modes include a curing mode for use during curing of the concrete; and a structure mode for use after curing of the concrete.

In one embodiment, the controller is configured to significantly reduce power consumption of the structure sensor device in the structure mode, compared to the curing mode.

In one embodiment, the controller is configured to cause the monitoring data to be transmitted at shorter intervals in the curing mode and longer intervals in the structure mode.

In one embodiment, the controller is configured to, in the curing mode, cause the monitoring data to be transmitted at intervals of one of: a day; a number of hours; an hour; a number of minutes; and 30 minutes.

In one embodiment, the controller is configured to, in the structure mode, cause the monitoring data to be transmitted at intervals of one of: a day; a number of days; a week; and a number of weeks.

In one embodiment, the controller is configured to, in the curing mode, generate the monitoring data using sensor data obtained from the temperature sensor and the moisture sensor.

In one embodiment, the sensors include an accelerometer and the controller is configured to, in the structure mode, generate the monitoring data using sensor data obtained from at least the accelerometer.

In one embodiment, the controller is configured to, in the structure mode, generate the monitoring data additionally using sensor data obtained from the temperature sensor and the moisture sensor.

In one embodiment, the controller is configured to, in the structure mode: determine whether an alert condition exists based on sensor data obtained from the accelerometer; in the event of determining that an alert condition exists, generating alert data; and causing the alert data to be transmitted to the gateway device using the wireless transceiver.

In one embodiment, the controller is configured to initially operate under the curing mode and switch to the structure mode after a predetermined period of time.

In one embodiment, the predetermined period of time is 60 days.

In one embodiment, the wireless transceiver is configured for wirelessly communicating with the gateway device using a LoRa wireless communication protocol.

In one embodiment, the wireless transceiver is configured for wirelessly communicating with the gateway device in a frequency range of at least one of: between 433 MHz and 928 MHz; between 902 MHz and 928 MHz; and between 915 MHz and 928 MHz.

In one embodiment, the wireless transceiver is configured for wirelessly communicating with another structure sensor device.

In one embodiment, the controller is configured to cause the monitoring data to be transmitted to the gateway device via the other structure sensor device.

In one embodiment, the moisture sensor is configured to measure a relative humidity within the structure.

In one embodiment, the structure sensor device further includes two probes, the controller being configured to measure electrical signals in the two probes and determine a distance between the two probes based on the measured electrical signals, to thereby allow shrinkage of the structure to be monitored.

In one embodiment, the structure sensor device includes a magnetic switch that can be activated by bringing an external magnet into proximity of the magnetic switch, and the structure sensor device is configured to respond to activation of the magnetic switch by at least one of: switching on the structure sensor device; switching off the structure sensor device; switching between operation modes; and causing the structure sensor device to be wirelessly paired with a gateway device.

In one embodiment, the structure sensor device is configured to be remotely switched off using the gateway device.

In one embodiment, the controller is configured to conserve power by alternating between a sleep state and an awake state at predetermined intervals, and at least the wireless transceiver is deactivated in the sleep state.

In one embodiment, the structure sensor device includes a power supply including a battery and a super capacitor.

In one embodiment, the battery is a Lithium Thionyl Chloride battery.

In one embodiment, the battery is used to provide a steady low operating current and the super capacitor is used to provide high short duration pulse currents for powering the wireless transceiver transmitting the monitoring data at the predetermined intervals.

In one embodiment, the housing is waterproof.

In one embodiment, the housing does not include any: external control inputs; external electrical connections; and external antennae.

In one embodiment, the housing includes at least one of: fastening points for attachment of separate fastening devices; holes for attachment of cable ties; and integral fastening devices.

In another broad form, an aspect of the present invention seeks to provide a system for monitoring physical properties within one or more bodies of structure, the system including: a plurality of structure sensor devices as described above, each structure sensor device being embedded inside a body of structure in use; and a gateway device located proximate to one or more bodies of structure in use, the gateway device including: a gateway wireless transceiver for wirelessly communicating with the plurality of structure sensor devices, the gateway wireless transceiver being configured for wirelessly communicating at frequencies of less than 1 GHz; a gateway wireless network interface for wirelessly communicating with a processing system via a wireless communications network; and a gateway processing system configured to receive monitoring data from the plurality of structure sensor devices, and transmit at least some of the monitoring data to the processing system via the wireless communications network.

In one embodiment, the gateway wireless network interface includes a wireless modem for wirelessly communicating with a server processing system via the Internet.

In one embodiment, the server processing system is configured to allow a remote user device to access monitoring data from the server processing system via the Internet.

In one embodiment, the server processing system is configured to allow the remote user device to access the monitoring data using one of a web portal and an application programming interface.

In one embodiment, the gateway processing system is configured to: receive alert data from one of the plurality of structure sensor devices and relay the alert data to the server processing system; and the server processing system is configured to, upon receipt of the alert data, generate an alert notification, and transmit the alert notification to the remote user device.

In one embodiment, the server processing system is configured to: receive, from the remote user device, a user command for controlling the operation of a selected one or more of the plurality of structure sensor devices; and transmit the user command to the gateway device, and the gateway processing system is configured to transmit structure sensor device control signals to the selected one or more of the plurality of structure sensor devices.

In one embodiment, the server processing system is configured to determine an estimated strength of the structure based on received monitoring data.

In one embodiment, the server processing system is configured to: determine whether the estimated strength of the structure has reached a predetermined threshold; and in the event of a successful determination, generate a strength notification; and transmit the strength notification to the remote user device.

In one embodiment, the server processing system includes a database for storing received monitoring data.

In one embodiment, the gateway wireless network interface includes a wireless access point for allowing a local user device to wirelessly connect to the gateway device to allow the local user device to access monitoring data directly from the gateway device.

It will be appreciated that the broad forms of the invention and their respective features can be used in conjunction, interchangeably and/or independently, and reference to separate broad forms is not intended to be limiting.

### BRIEF DESCRIPTION OF THE SEVERAL VIEWS OF THE DRAWINGS

Various examples and embodiments of the present invention will now be described with reference to the accompanying drawings.

FIG. 1 is a schematic view of a diagram of an example of a structure sensor device for use in monitoring physical properties within a body of concrete.

FIG. 2 is a schematic view of a diagram of an example of a system for monitoring physical properties within a body of concrete including a plurality of the concrete sensor devices of FIG. 1.

FIG. 3 is a schematic view of a diagram of an example of a gateway device of the system of FIG. 2.

FIG. 4 is a schematic view of a diagram of an example of a server processing system of the system of FIG. 2.

FIG. 5 is a schematic view of a diagram of an example of a user device of the system of FIG. 2.

FIG. 6 is a schematic view of a diagram of hardware elements of another example of a structure sensor device.

FIG. 7A is a perspective view of another example of a structure sensor device.

FIG. 7B is a perspective view of the structure sensor device of 7 A with a cover portion of its housing removed.

FIG. 7C is a first exploded perspective view of the structure sensor device of 7A.

FIG. 7D is a second exploded perspective view of the structure sensor device of 7A.

### DETAILED DESCRIPTION OF THE INVENTION

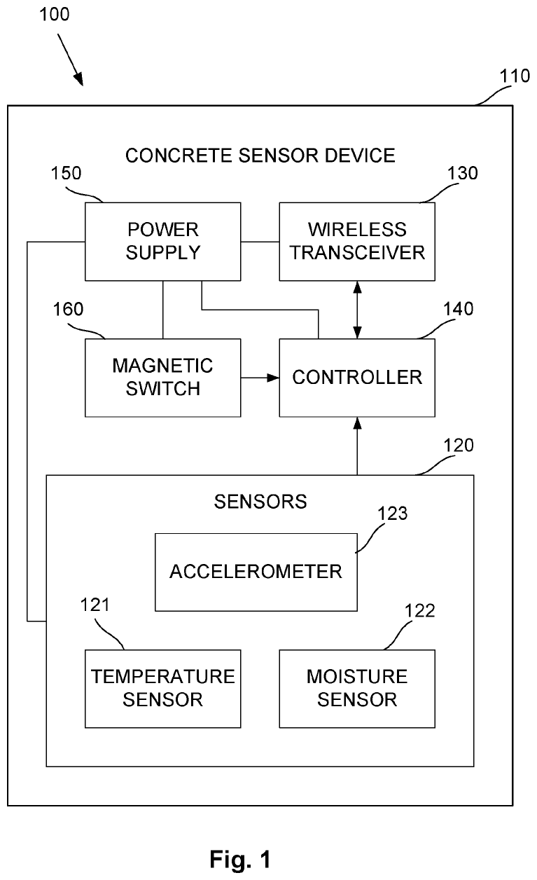

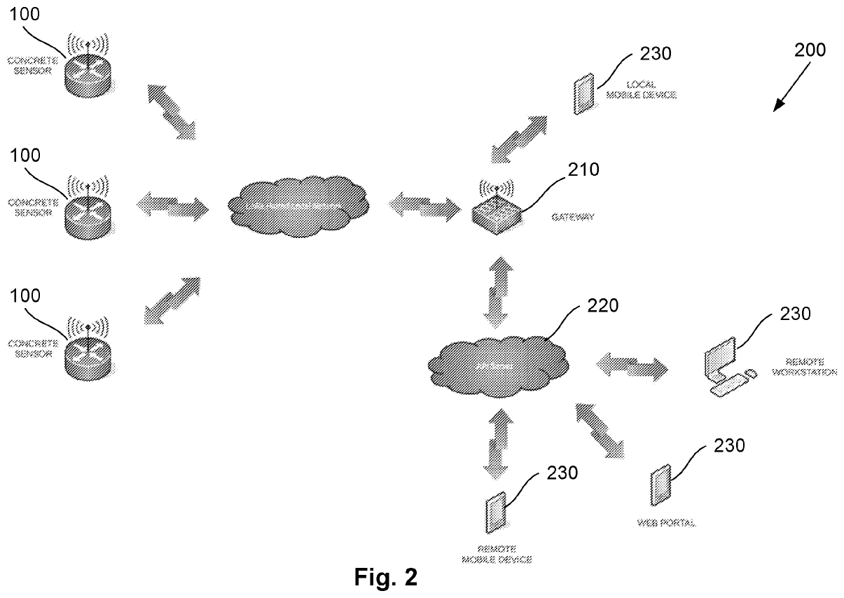

An example of a concrete sensor device 100 for use in monitoring physical properties within a body of concrete will now be described with reference to FIG. 1, which shows a schematic representation of elements of the concrete sensor device 100, and FIG. 2, which shows a system 200 including a plurality of the concrete sensor devices 100.

The concrete sensor device 100 is configured to be embedded inside the body of concrete in use, and generally includes a sealed housing 110, sensors 120 for measuring physical properties within the body of concrete, a wireless transceiver 130 for wirelessly communicating with a gateway device 210 (as shown in the system 200 of FIG. 2) that is located outside of the body of concrete, and a controller 140.

The controller 140 is particularly configured to obtain sensor data from the sensors 120, generate monitoring data using at least some of the sensor data, and cause the monitoring data to be transmitted to the gateway device 210 using the wireless transceiver 130, at predetermined intervals.

The controller 140 can be of any appropriate form, but in one example includes at least one microprocessor, a memory, and an external interface, which may be interconnected by a bus. In this case, the external interface is connected to the sensors 120 and the wireless transceiver 130. In use, the microprocessor executes instructions in the form of applications software stored in the memory to allow the required processes to be performed. Accordingly, it will be appreciated that the controller 140 may be formed from any suitable processing system arrangement and could include any electronic processing device such as a microcontroller unit, microchip processor, logic gate configuration, firmware optionally associated with implementing logic such as an FPGA (Field Programmable Gate Array), or any other electronic device, system or arrangement.

As will be discussed in further detail below, and with regard to the example of the system 200 shown in FIG. 2, the gateway device 210 may in turn relay received monitoring data to a server processing system 220 for facilitating access to the monitoring data by remote user devices 230, via a communications network such as the Internet. In some examples, the server processing system 220 may be provided in the form of a cloud-based serve. As indicated in the example of the system shown in FIG. 2, the server processing system 220 may be an Application Programming Interface (API) Server, however this is not essential.

The wireless transceiver 130 is preferably configured for wirelessly communicating at frequencies of less than 1 GHz. It will be appreciated that frequencies of less than 1 GHz are significantly lower than the frequencies of commonly used wireless networking protocols such as Bluetooth (which uses a 2.4 GHz frequency band) or Wi-Fi (which typically uses 2.4 GHz or 5 GHz frequency bands). It has been found that lower, sub-1 GHz frequencies provide superior signal penetration through concrete compared to higher frequency technologies including Bluetooth and Wi-Fi.

In some embodiments, the wireless transceiver 130 may be configured for wirelessly communicating with the gateway device 210 using a long range wireless data communication technology, particularly using the LoRa wireless communication protocol. Depending on the particular implementation, this can involve the use of frequencies between 433 MHz and 928 MHz. Preferably, this will involve the use of frequencies between 902 MHz and 928 MHz, and even more preferably, frequencies between 915 MHz and 928 MHz, which correspond to the LoRaWAN frequency plan used in Australia. Signal penetration tests through concrete have confirmed that the performance of LoRa communications far exceeds that of Bluetooth (which has been traditionally used in many existing concrete sensor systems) and achieves long range signal penetration which is ideal for use on construction sites that may contain many obstructions.

It will be appreciated that the use of a relatively low frequency range compared to other conventional wireless communication protocols as discussed above enables communications from multiple concrete sensor devices 100 embedded inside the concrete to a gateway device 210 located outside the concrete, without the need for any external transmitters or repeaters. In contrast, as mentioned previously, conventional concrete sensor systems have typically required the use of transmitter probes extending from the embedded device to a location outside of the concrete or short range Bluetooth/Wi-Fi communications to repeaters located in close proximity to the embedded devices. Conventional concrete sensors also typically required manual interrogation of individual sensors using a local user device, as opposed to the ability to access monitoring data via a server processing system using a remote user device as in the above described techniques.

In some implementations, the wireless transceiver 130 may optionally be configured for wirelessly communicating with other concrete sensor devices 100. Accordingly, the controller 140 may be configured to cause the monitoring data to be transmitted to the gateway device 210 via one or more other concrete sensor devices 100. It will be appreciated that this can enable the use of a mesh network topology in which the concrete sensor devices 100 can relay data from other concrete sensor devices 100 to the gateway device 210. This can assist in signal connectivity, to effectively increase the range in which concrete sensor devices 100 can be separated from the gateway device 210. However, this is not essential.

The sensors 120 typically include at least a temperature sensor 121 and a moisture sensor 122. The moisture sensor 122 is preferably in the form of a relative humidity sensor although other forms of moisture sensors may be used. In some embodiments, an integrated temperature and humidity sensor may be provided. In any event, it will be appreciated that temperature and moisture measurements from within concrete can be used to determine an estimated strength of the concrete using known concrete strength estimation techniques. Accordingly, the monitoring data that is generated using sensor data obtained from the temperature and moisture sensors 121, 122 and transmitted to the gateway device 210 at predetermined intervals can be used to progressively monitor the strength of the concrete, which can be especially important during curing of the concrete after pouring. Users can conveniently access the monitoring data using remote user devices 230 to monitor physical properties within the body of concrete in near real-time from remote locations.

In preferred embodiments, the sensors 120 may further include an accelerometer 123. It will be appreciated that the addition of the accelerometer 123 to the concrete sensor device 100 can enable the collection of further measurements of the structural performance of the concrete, which can be especially beneficial for long term structural monitoring. Accordingly, the concrete sensor device 100 can provide valuable monitoring functionalities extending significantly beyond the initial curing process of the concrete.

In such embodiments, the controller 140 may be configured to use sensor data obtained from the accelerometer 123 to determine at least one of a tilt angle of the concrete sensor device 100, stiffness of the concrete, and vibration in the concrete. These types of measurements can be used to allow users to monitor the ongoing performance of the structure, for instance by identifying any changes in tilt or stiffness. Moreover, as described further below, in some implementations the system 200 may be configured to generate a high priority alert if the structure is overloaded or deflecting more than specified.

Embodiments of the concrete sensor device 100 may be configured to operate under different operating modes, such that the controller 140 may be configured to generate the monitoring data in accordance with a current operating mode and also cause the monitoring data to be transmitted to the gateway device 210 at intervals selected in accordance with the current operating mode. It will be appreciated that the different operating modes may involve different operational procedures to reflect, for example, different stages of throughout the construction and ongoing operation of the body of concrete.

In preferred embodiments of the concrete sensor device 100, the operating modes include a curing mode for use during curing of the concrete, and a structure mode for use after curing of the concrete. Typically, the behaviour of the controller 140 for controlling the operation of other elements of the concrete sensor device 100 will vary depending on the operating mode. In some examples, the controller 140 may be configured to significantly reduce power consumption of the concrete sensor device 100 when in the structure monitoring mode, compared to in the concrete curing mode.

It will be appreciated that during the initial concrete curing period, which may extend for durations on the order of two months, it may be desirable to operate in a relatively high powered mode with the concrete sensor device 100 frequently collecting and transmitting monitoring data to allow users to access more up to date information regarding the strength and other properties of the concrete throughout the curing process. On the other hand, once the concrete has cured to a sufficient level of maturity to develop its full design strength, the concrete sensor device 100 may switch to a relatively low powered mode that will allow the ongoing performance of the structure to be monitored with lower power demands.

In some implementations, the controller 140 may be configured to cause the monitoring data to be transmitted at shorter intervals in the concrete curing mode and longer intervals in the structure monitoring mode. It will be appreciated that the use of longer intervals between transmissions will significantly reduce the power demands due to the reduced time the wireless transceiver 130 will be actively transmitting monitoring data to the gateway device 210.

In the curing mode, the intervals may be on the order of an hour. For example, the intervals may be selected as a day, but more typically a number of hours, an hour, or a number of minutes. The specific interval timing will depend on a trade-off between the potential drain on power reserves and the currency of the monitoring data that will be available to users. In one particular implementation, the monitoring data will be transmitted to the gateway device at intervals of 30 minutes.

In contrast, in the structure mode, the intervals may be on the order of days or even weeks, since the currency of the monitoring data will be less critical for the ongoing monitoring of the structural performance compared to during the concrete curing process. For example, the intervals may be selected as a day, a number of days, a week, or even a number of weeks. In one particular implementation, the monitoring data will be transmitted to the gateway device at weekly intervals.

However, it should be appreciated that the above discussed intervals are not essential and may be reconfigurable depending on user preferences.

The concrete sensor device 100 will typically include an internal power supply 150 for powering the concrete sensor device 100 for its entire operational life. In some examples, the power supply 150 may include a battery and a super capacitor. In one specific implementation, the battery may be a long life, high capacity Lithium Thionyl Chloride battery, which can be used in conjunction with the super capacitor to provide extended in-service lifetime of months, or even years. The battery may be used to provide a steady low operating current and the super capacitor may be used to provide high short duration pulse currents for powering the wireless transceiver 130 transmitting the monitoring data at the predetermined intervals.

In some examples, the controller 140 may be configured to conserve power by selectively switching between a sleep state and an awake state, and this may also be performed at predetermined intervals, which may or may not coincide with the above discussed intervals for transmission of monitoring data. The controller 140 may selectively activate or deactivate particular elements of the concrete sensor device 100 depending on the sleep or awake state. For instance, at least the wireless transceiver 130 may be deactivated in the sleep state to significantly reduce power demands. The controller 140 may be configured to enter the sleep state for longer intervals in the structure mode compared to in the curing mode.

As mentioned above, the controller 140 may be configured to generate the monitoring data in accordance with the operating mode. It will be appreciated that different data may be of interest to users in the different modes. For instance, in the curing mode, the monitoring data may be generated using sensor data obtained from the temperature sensor 121 and the moisture sensor 122 only. On the other hand, in the structure mode, the monitoring data may be generated using sensor data obtained from only the accelerometer 123, but typically the monitoring data will still include some of the sensor data obtained from the temperature sensor 121 and the moisture sensor 122 to also allow ongoing monitoring of temperature and moisture levels.

It should also be appreciated that the content and quantity of the monitoring data may vary depending on the current operating mode, such as by incorporating the full set of sensor data in the curing mode versus an abridged set of sensor data or only indicators of the sensor data in the structure mode. This can also be used to conserve power in the structure mode compared to in the curing mode.

To account for the typically longer intervals between transmissions of monitoring data in the structure mode, the concrete sensor device 100 may also be capable of transmitting alerts outside of scheduled transmission intervals. For example, in the structure monitoring mode, the controller 140 may determine whether an alert condition exists based on sensor data obtained from the accelerometer 123, and in the event of determining that an alert condition exists, generate alert data and cause the alert data to be transmitted to the gateway device 210 using the wireless transceiver 130. Thus, users may be alerted to structural abnormalities such as when structure is overloaded or deflecting more than specified, without having to wait for the next interval to elapse.

Typically, the controller 140 will be configured to initially operate under the curing mode and switch to the structure mode after a predetermined period of time. Typically, the time period for switching modes will be based on the expected curing time of the concrete, and in one specific example the predetermined period of time is 60 days.

As mentioned above, the moisture sensor may be particularly configured to measure a relative humidity within the concrete. In preferred embodiments, the relative humidity may be provided as a percentage value, which can further allow users to determine whether floor finishes can be installed in a timely manner without risk of damaging flooring systems.

In some embodiments, the concrete sensor device 100 may also be optionally adapted for monitoring shrinkage of the concrete. For example, the concrete sensor device may include two probes (not shown), and the controller 140 may be configured to measure electrical signals in the two probes and determine a distance between the two probes based on the measured electrical signals, to thereby allow shrinkage of the concrete to be monitored.

As mentioned above, the concrete sensor device 100 includes a sealed housing 110. Typically, the housing 110 will have a waterproof construction to ensure the internal elements are not exposed to potentially damaging moisture from within the concrete. Preferably, to aid in the effective sealing, the housing 110 may be constructed so that it does not include any external control inputs, external electrical connections or any external antennae. However, it may still be desirable to provide functionalities for allowing a user to control certain operations of the concrete sensor device 100, such as to switch on the concrete sensor device 100 after a period of storage before use, rather than having the concrete sensor device 100 be always on and draining power unnecessarily.

In order to achieve this, in some implementations, the concrete sensor device 100 may include a magnetic switch 160 (as shown in FIG. 1) that can be activated by bringing an external magnet into proximity of the magnetic switch 160, for example by momentarily placing a permanent magnet on the outside of the housing 110. It will be appreciated that this can allow user inputs to be provided to the concrete sensor device 100 without requiring any physical external control inputs such as buttons, switches, or the like. The concrete sensor device 100 may be configured to respond to activation of the magnetic switch 160 by at least one of switching on the concrete sensor device 100, switching off the concrete sensor device 100, switching between operation modes, and causing the concrete sensor device 100 to be wirelessly paired with a gateway device 210.

In some embodiments, different control inputs may effectively be provided depending on the current state of the concrete sensor device 100 and potentially the duration of activation by the external magnet. For example, if the concrete sensor device 100 is currently switched off, the concrete sensor device 100 may be switched on by holding the external magnet in proximity for a short predetermined period of time, such as one second. Once the concrete sensor device 100 has been switched on, it may be switched off again by holding the external magnet in proximity for a longer predetermined period of time, such as six seconds. Additionally or alternatively, the concrete sensor device 100 may be configured to be remotely switched off using the gateway device 210.

In some examples, the concrete sensor device 100 may automatically attempt to pair with a nearby gateway device 210 as soon as it is switched on. However, it may also be possible to manually initiate pairing using activation of the magnetic switch.

Depending on the particular implementation, the housing 110 may also include a range of different structural features for facilitating installation of the concrete sensor device 100 within the body of concrete. In particular, the housing 110 may be adapted to allow the concrete sensor device 100 to be more easily fastened to concrete reinforcement structures before the concrete is poured, so that the concrete sensor device 100 can be more reliably embedded in a desired position within the body of concrete after pouring.

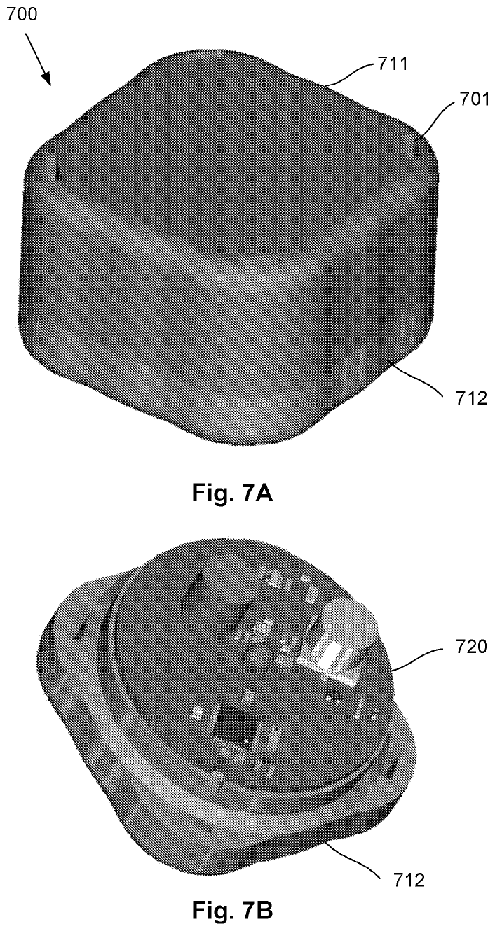

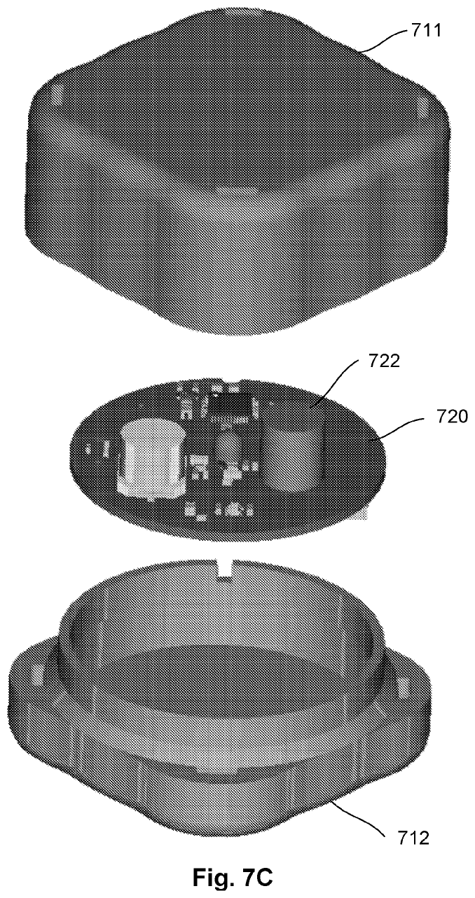

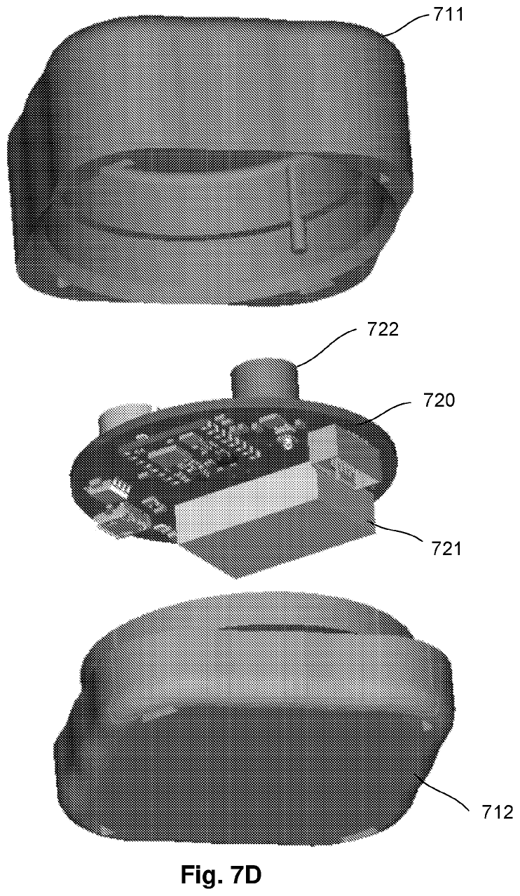

For example, the housing 110 may include fastening points for attachment of separate fastening devices, holes specifically provided for attachment of cable ties, integral fastening devices such as straps, or the like. An example of a suitable housing 110 design is shown in FIGS. 7A to 7D and will be described in further detail in due course.

It will be appreciated that a system 200 for monitoring physical properties within one or more bodies of concrete will generally include a plurality of concrete sensor devices 100 as described above. Each concrete sensor device 100 will be embedded inside a body of concrete 200 in use, and it should be appreciated that the concrete sensor devices 100 may be embedded in distributed locations throughout the same body of concrete, or may even be embedded in different bodies of concrete, such as different concrete floor slabs or other concrete structures within a building or other structure constructed using concrete.

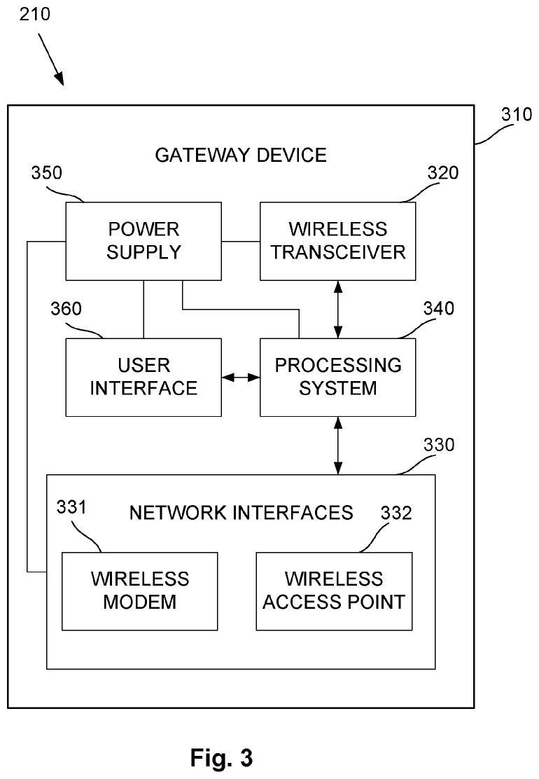

The system 200 will also include a gateway device 210 located proximate to one or more bodies of concrete 200 in use. Functional elements of an example of a gateway device 210 are shown in FIG. 3. Typically, the gateway device 210 includes a gateway wireless transceiver 320 for wirelessly communicating with the plurality of concrete sensor devices 100, a gateway wireless network interface 330 for wirelessly communicating with a processing system, such as the server processing system 220, via a wireless communications network, and a gateway processing system 340. The gateway device 210 may also include an internal power supply 350 as shown, although the gateway device 210 could alternatively be connected to an external power source.

The gateway processing system 340 is configured to receive monitoring data from the plurality of concrete sensor devices 100, and transmit at least some of the monitoring data to the server processing system 220 (or other processing systems) via the wireless communications network.

The gateway processing system 340 can be of any appropriate form, but in one example includes at least one microprocessor, a memory and an external interface, which may be interconnected via a bus. In this case, the external interface is connected to the wireless gateway transceiver 320, the gateway wireless network interface, and could additionally be used to connect the gateway processing system 340 to an optional user interface 360 such as a touch screen as described further below.

In use, the microprocessor executes instructions in the form of applications software stored in the memory to allow the required processes to be performed. Accordingly, it will be appreciated that the gateway processing system 340 may be formed from any suitable processing system arrangement and could include any electronic processing device such as a microprocessor, microchip processor, logic gate configuration, firmware optionally associated with implementing logic such as an FPGA (Field Programmable Gate Array), or any other electronic device, system or arrangement.

As per the wireless transceiver 130 of the concrete sensor device 100, the gateway wireless transceiver 320 is typically configured for wirelessly communicating at frequencies of less than 1 GHz, and in some implementations this may involve using the LoRa wireless communication protocol.

The gateway wireless network interface 330 may include a wireless modem for wirelessly communicating with a server processing system 220 via the Internet 201. In one example, the wireless modem may be a 4G wireless modem. In any event, the gateway device 210 will be capable of connecting to the Internet to thereby transmit monitoring data to the server processing system 220. It therefore acts as a data gateway between the local concrete sensor device network and the Internet.

As mentioned previously, the server processing system 220 may be configured to allow a remote user device 230 to access monitoring data from the server processing system 220 via the Internet 201, as shown in FIG. 2. For example, this access may be facilitated using a web portal hosted by the server processing system 220 or using an application programming interface (API).

In one specific implementation, the server processing system 220 may be in the form of an Internet connected web server providing an API for the facilitating the transfer of monitoring data received from the concrete sensor devices 100. In addition to facilitating the data connection to the gateway device 210, the API can also be utilised by various other systems such as mobile or desktop applications, and online web portals. The server processing system 220 may additionally include a database for storing received monitoring data, and allowing this to be queried, via the API for example.

As mentioned previously, the concrete sensor devices 100 may be used to generate and transmit alert data under certain conditions, such as to alert users of structural issues outside of predetermined intervals for transmitting monitoring data. These alerts may be facilitated by the system 200, by having the gateway processing system 210 configured to receive alert data from one of the plurality of concrete sensor devices 100 and relay the alert data to the server processing system 220. In turn, the server processing system 220 may be configured to, upon receipt of the alert data, generate an alert notification and transmit the alert notification to the remote user device 230.

The system 200 may also enable the remote control of the concrete sensor devices 100, such as to selectively deactivate them or change their operation modes. In one example, the server processing system 220 may be configured to receive, from the remote user device 230, a user command for controlling the operation of a selected one or more of the plurality of concrete sensor devices 100, and subsequently transmit the user command to the gateway device. In turn, the gateway processing system 210 may be configured to transmit suitable concrete sensor device control signals to the selected one or more of the plurality of concrete sensor devices 100.

As discussed previously, the concrete sensor devices 100 enable estimates of the strength of the concrete to be determined using the sensor data obtained from the temperature and moisture sensors. In preferred implementations, the server processing system 220 may be configured to determine an estimated strength of the concrete based on received monitoring data, to thereby allow this estimated strength information to be provided to users without requiring further processing.

In some examples, the server processing system 220 may be further configured to automatically monitor the estimated strength of the concrete during curing and alert the user at particular milestones in the curing process. For instance, the server processing system 220 may be configured to determine whether the estimated strength of the concrete has reached a predetermined threshold, and in the event of a successful determination, generate a strength notification and transmit the strength notification to the remote user device.

In some implementations, the gateway wireless network interface may also include a wireless access point for allowing a local user device 230 to wirelessly connect to the gateway device 210, to thereby allow the local user device 230 to access monitoring data directly from the gateway device 210. For example, the wireless access point may be provided as a Wi-Fi access point, whereby local user devices 230 such as on-site mobile phones and laptops can connect to the gateway device 210 by the Wi-Fi wireless communication protocol.

In some embodiments, the gateway device 210 may include an integrated web server for facilitating direct access to the monitoring data using local user devices 230. This can allow local user devices 230 to access monitoring data received from the concrete sensor devices 100 without requiring Internet access.

Optionally, the gateway device 210 may also include a touch screen display with a simple Graphical User Interface (GUI) to allow end users to access further functionalities, such as to manually pair the gateway device 210 with concrete sensor devices 100 and to confirm that concrete sensor devices 100 have been successfully paired before they are embedded within the concrete.

In the example of the system 200 depicted in shown in FIG. 2, the system includes a cloud-based server processing system 220 and a number of user devices 230 interconnected via the Internet. It will be appreciated that this system configuration is for the purpose of example only, and in practice the server processing system 230 and the user devices 230 can communicate via any appropriate mechanism, such as via wired or wireless connections, including, but not limited to mobile networks, private networks, such as an 802.11 networks, the Internet, LANs, WANs, or the like, as well as via direct or point-to-point connections, or the like. The nature of the server processing system 220 and user devices 230 and their functionality will vary depending on their particular requirements.

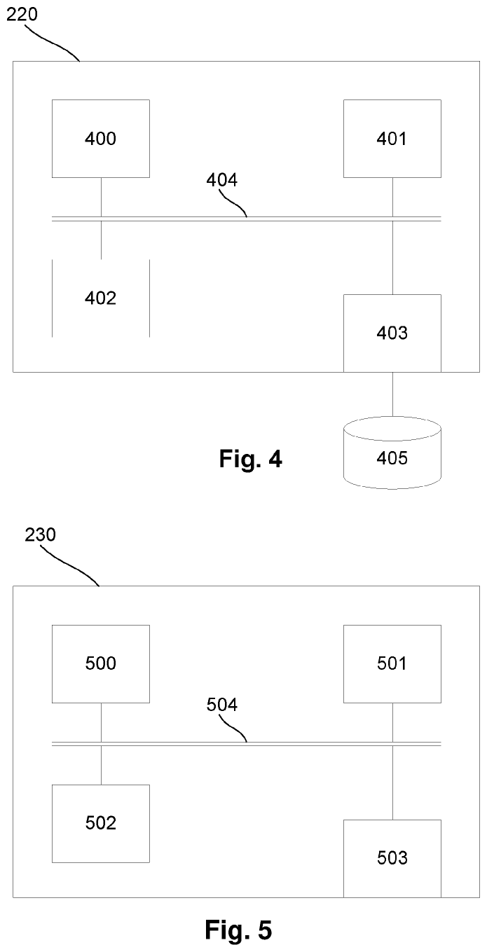

An example of a suitable server processing system 220 is shown in FIG. 4. In this example, the server processing system 220 includes an electronic processing device, such as at least one microprocessor 400, a memory 401, an optional input/output device 402, such as a keyboard and/or display, and an external interface 403, interconnected via a bus 404 as shown. In this example, the external interface 403 can be utilised for connecting the processing system 401 to peripheral devices, such as communications networks, databases 405, other storage devices, or the like. Although a single external interface 403 is shown, this is for the purpose of example only, and in practice multiple interfaces using various methods (e.g. Ethernet, serial, USB, wireless or the like) may be provided.

In use, the microprocessor 400 executes instructions in the form of applications software stored in the memory 401 to perform required processes, such as communicating with the gateway device 210 and user devices 230. Thus, actions performed by a server processing system 220 are performed by the processor 400 in accordance with instructions stored as applications software in the memory 401 and/or input commands received via the I/O device 402, or commands received from user devices 230. The applications software may include one or more software modules, and may be executed in a suitable execution environment, such as an operating system environment, or the like.

Accordingly, it will be appreciated that the server processing system 220 may be formed from any suitable processing system, such as a suitably programmed computer system, PC, web server, network server, or the like. In one particular example, the processing system 220 is a standard processing system such as a 32-bit or 64-bit Intel Architecture based processing system, which executes software applications stored on non-volatile (e.g., hard disk) storage, although this is not essential. It will also be understood that the server processing system 220 could be or could include any electronic processing device such as a microprocessor, microchip processor, logic gate configuration, firmware optionally associated with implementing logic such as an FPGA (Field Programmable Gate Array), or any other electronic device, system or arrangement. In preferred implementations, the server processing system 220 will be provided using processing systems provided as part of a cloud based environment.

As shown in FIG. 5, in one example, the user device 230 includes an electronic processing device, such as at least one microprocessor 500, a memory 501, an input/output device 502, such as a keyboard and/or display, and an external interface 503, interconnected via a bus 504 as shown. In this example, the external interface 503 can be utilised for connecting the user device 230 to peripheral devices, such as the communications networks, databases, other storage devices, or the like. Although a single external interface 503 is shown, this is for the purpose of example only, and in practice multiple interfaces using various methods (e.g. Ethernet, serial, USB, wireless or the like) may be provided.

In use, the microprocessor 500 executes instructions in the form of applications software stored in the memory 501 to perform required processes, for example to allow communication with the server processing system 220. Thus, actions performed by a user device 230 are performed by the processor 501 in accordance with instructions stored as applications software in the memory 502 and/or input commands received from a user via the I/O device 503. The applications software may include one or more software modules, and may be executed in a suitable execution environment, such as an operating system environment, or the like.

Accordingly, it will be appreciated that the user devices 230 may be formed from any suitable processing system, such as a suitably programmed PC, Internet terminal, lap-top, hand-held PC, smart phone, PDA, tablet, or the like. Thus, in one example, the user device 230 is a standard processing system such as a 32-bit or 64-bit Intel Architecture based processing system, which executes software applications stored on non-volatile (e.g., hard disk) storage, although this is not essential. However, it will also be understood that the user device 230 can be any electronic processing device such as a microprocessor, microchip processor, logic gate configuration, firmware optionally associated with implementing logic such as an FPGA (Field Programmable Gate Array), or any other electronic device, system or arrangement.

In view of the above, it will be appreciated that access to monitoring data may be administered by the server processing system 220 and interaction by a user may be via a user device 230. The user may interact with the server processing system 220 via a GUI (Graphical User Interface), or the like, presented on the user device 230, and in one particular example via a browser application that displays webpages hosted by the server processing system 220. However, it will be appreciated that the above described configuration is not essential, and numerous other configurations may be used.

Further optional features of preferred embodiments of concrete sensor devices will now be described.

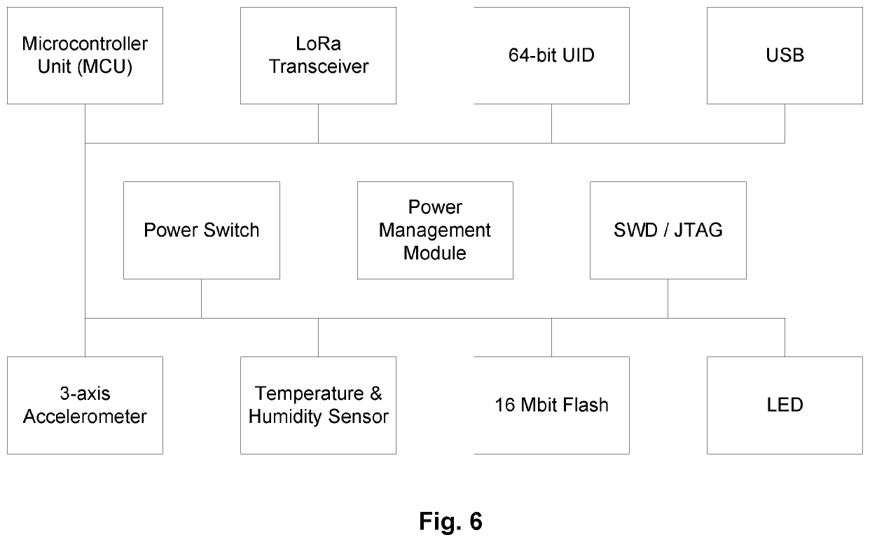

Specific hardware modules that may be provided in example implementation of a concrete sensor device are shown in FIG. 6. In this example, the different hardware modules are interconnected by a bus. In accordance with the previous examples, this example of the concrete sensor device includes a controller 140 in the form of a Microcontroller Unit (MCU), a wireless transceiver 130 in the form of a LoRa Transceiver module, and sensors 120 including an integrated Temperature & Humidity Sensor and a 3-axis Accelerometer. In addition, this example of the concrete sensor device includes a 16 Mbit Flash module for data storage, a Light Emitting Diode (LED) module for providing externally visible indicators of operational status or the like, a 64-bit Unique Identifier (UID) module for providing a unique identifier for networking purposes, a Universal Serial Bus (USB) module for internal connectivity, a Serial Wire Debug (SWD)/Joint Test Action Group (JTAG) module for debugging/testing, along with a Power Switch and a Power Management Module.

Physical construction details of another example of a concrete sensor device 700 are shown in FIGS. 7A to 7D. With regard to FIG. 7A, it will be seen that the housing 110 of this example of the concrete sensor device 700 includes a cover portion 711 and a base portion 712. The cover and base portions 711, 712 have a rounded square shape and cable tie holes extend through corner regions thereof to facilitate attachment by cable ties of the concrete sensor device 700 to reinforcement structures before concrete is poured. Otherwise, the concrete sensor device 700 is generally devoid of external physical features.

The cover and base portions 711, 712 are sealed together to enclose the internal hardware elements of the concrete sensor device 700, which can be seen in FIGS. 7B to 7D. As shown in FIG. 7B, the concrete sensor device 700 includes a Printed Circuit Board (PCB) 720 upon which are mounted electrical components for providing the above discussed functionalities of the concrete sensor device 700, such as Integrated Circuits (ICs) including the controller 140 and discrete components including the above mentioned battery 721 and super capacitor 722 of the power supply 150.

A detailed functional description of an example implementation of the concrete sensor device and a system using a plurality of the concrete sensor devices will now be provided, to provide further explanation of preferred and optional implementation features.

The concrete sensor device is an embedded wireless product, which accurately measures and transmits temperature and relative humidity data while fully interred within a curing concrete structure. This data is collected locally by a wireless gateway device and then forwarded via calls to a server processing system in the form of an API Server. Other systems such as mobile, desktop and web applications can then access this data via the same API, presenting it to end users for various purposes such as curing progress monitoring, data collection etc.

The system includes four key sub-modules, namely concrete sensor devices, a wireless network, a gateway device, and a server processing system.

In preferred implementations, the concrete sensor device has the following key technical features, which will be discussed in further detail below in turn:
1. Very Low Power Operation;
2. Long Range Wireless Communications;
3. Precision Temperature Sensor;
4. Precision Relative Humidity Sensor;
5. 3 -Axis Accelerometer;
6. Magnetic Switch Activation/Deactivation;
7. Fully Interred Operation; and
8. Small Physical Size.

With regard to the Very Low Power Operation feature, the concrete sensor device has an on-board power management system that can reduce total power to less than 16 micro amps in “sleep” mode and then “wake” at programmable intervals to record and transmit measurements to the local gateway device. With hourly “sleep” intervals, it is calculated that the sensor can continue to transmit for up to 12 months from a single charged 3.7V/750 mAh Lithium Thionyl Chloride battery cell.

As far as the Long Range Wireless Communications feature is concerned, by using the LoRa wireless communications protocol, data can be transmitted wirelessly through curing concrete for up to several hundred metres even within a cluttered building site and through multiple floors of a high-rise building.

The concrete sensor device incorporates a Precision Temperature Sensor providing ±0.4° C. accuracy over a temperature range of −10 to 85° C. The ASTM Standard C1074 provides a method to estimate the in-place strength of concrete from the recorded temperature to allow the start of critical construction activities such as: (1) removal of formwork and reshoring; (2) post-tensioning of tendons; (3) termination of cold weather protection; and (4) opening of roadways to traffic.

The concrete sensor device also incorporates a Precision Relative Humidity Sensor providing ±3% RH accuracy over a 0-80% RH range and with total operating range of 0-100% RH. The ASTM Standard F2170 covers the standard test method for determining relative humidity in concrete floor slabs using in situ probes.

The concrete sensor device includes a 3-Axis Accelerometer for allowing ongoing monitoring of the performance of the concrete structure. The accelerometer will allow identification of any changes in stiffness or tilt, which can be used to trigger user alerts if the structure is overloaded or deflecting more than specified.

The concrete sensor device is adapted to allow Magnetic Switch Activation/Deactivation. An advanced magnetic relay switch together with an ultra-low leakage circuit provides remote magnetic activation and deactivation of the sensor. Users can power on the sealed sensor unit by briefly applying an external magnet. The unit can then subsequently be switched off by applying the external magnet for several seconds. This eliminates the need for any external switches and increases the shelf life of a unit with a charged battery.

With regard to the Fully Interred Operation feature, the concrete sensor device is designed to be fully interred within the curing concrete. There are no external connections or antennae and the unit enclosure provides integrated fastening points for cable ties to facilitate its attachment to buried reinforcement etc. This greatly simplifies the installation of the devices. The concrete sensor device is able to transmit through at least 100 mm of curing concrete.

Finally, the concrete sensor device has a Small Physical Size of 55×55×35 mm, allowing it be located in virtually any concrete structure.

The wireless network has the following key technical features, which are expanded upon below:
1. Low Power Operation and Long Range
2. Adjustable Power and Range
3. High Penetration
4. Globally Unique Identifiers
5. Automatic Pairing

The Low Power Operation feature is facilitated by the wireless network utilising the LoRa wireless modulation system, which is characterised by very long range low power transmission.

The Adjustable Power and Range feature is provided because the LoRa protocol allows transmitters to trade off power with range. The Range, Data Rate, Bandwidth and Spreading Factor and may be configured to maximise the signal range and hence the penetration of the concrete slab.

With regard to the above mentioned feature of High Penetration, the preferred LoRa transmission frequency band of 915 MHz more effectively penetrates ground and water than higher frequencies such as the 2.4G Hz used by Bluetooth and Wi-Fi.

The wireless network uses Globally Unique Identifiers. Each concrete sensor device has a 64-bit globally unique identifier. This consists of a 24 bit Organisation Unique Identifier, (OUI) and a 40 bit Unique Identifier (UID). This means that every device can be uniquely identified across the entire world greatly simplifying the task of separating different site data.

Finally, the wireless network allows Automatic Pairing. When a concrete sensor device is first activated, it begins an automated pairing procedure looking for the nearest gateway device. Once communication is established with a pairing-enabled gateway device, the two devices complete a data handshake with each other, which provides both of them with the other's address. This information is recorded at both ends establishing a permanent data connection between the two.

Next, the gateway device has the following key technical features, as detailed further below:
1. LoRa Wireless Transceiver
2. 4G Wireless Modem
3. Wi-Fi Access Point
4. Integrated Web Server
5. Touch Screen

The gateway device has a LoRa Wireless Transceiver that acts as the pairing “master” for the concrete sensor device “slaves”. The gateway device's processing system can query the LoRa Wireless Transceiver for sensor data and other information from the concrete sensor devices.

The gateway device also has a 4G Wireless Modem, which allows it to connect to the Internet and thereby transmit the sensor data to the API Server. It therefore acts as a data “gateway” between the local sensor network and the internet.

The gateway device has a Wi-Fi access point, which allows it to connect to local devices such as on-site mobile phones and laptops.

The gateway device further provides an Integrated Web Server, which allows local devices such as on-site mobile phones and laptops to configure the gateway and to view the local sensor data without having to connect to the internet. This provides support for remote locations where internet access is unavailable.

The gateway device also has a Touch Screen with a simple Graphical User Interface, (GUI) to allow end user's to manually pair with devices and to confirm that devices have successfully paired before they are interred.

Finally, the API Server is an Internet connected web server providing a “RESTful” (Representational State Transfer) API for the global transfer of sensor data. In addition to the gateway device connections, the API can also be utilised by various other systems such as Mobile Applications, (Apps), Desktop Applications and online Web Portals.

It will be appreciated that the above examples provide a state of the art concrete sensor device that will be able to provide temperature, relative humidity and load stiffness data & monitoring in near real-time. The concrete sensor device may commence in a curing mode of operation, gathering real time data every 30 minutes without any human interaction via a gateway device back to a web portal that captures, outputs, collates and stores the data.

Once the curing process is complete (e.g. in 60 days), the concrete sensor device will switch to a structure mode of operation. This will operate on a low power setting that will monitor the ongoing performance of the structure. The accelerometer will be able to identify any changes in stiffness or tilt, which will be able to send the data through the web portal and also send a high priority alert if the structure is overloaded or deflecting more than specified.

Particular advantages of the concrete sensor device and associated system include the following. The concrete sensor device provides two different modes of operation, namely high powered data collecting mode for the Curing Process of 60 days and low powered data collecting mode for the ongoing monitoring of the structure. The concrete sensor device provides compressive strength, relative humidity and structure movement alert monitoring. The concrete sensor device will be able to be toggled on and off and also paired by using a magnetic switch at the top of the sensor. The concrete sensor device communicates using a low frequency range of 915-928 MHz to provide the signal strength and penetration required for cast in sensors to be efficient and economical. As mentioned previously, signal penetration tests through concrete, have shown that Wi-Fi or Bluetooth were inadequate options, whereas LoRa (at a frequency of 915-928 MHz) far exceeds Bluetooth and achieves long range signal penetration, which is ideal for use on construction sites that contain many obstructions.

Further benefits of preferred implementations of the concrete sensor device are as follows. The concrete sensor devices will provide relative humidity in a percentage so that floor finishes can be installed timely with no risk to damaging flooring systems. Also captured is near real-time data showing compressive strength in MPa of the concrete, along with ongoing monitoring data and alerts of any building movements (tilt) or change in stiffness. The concrete sensor devices can send all information to a website that can be accessed with login and passwords anywhere in the world to gain all the project information. The concrete sensor devices will store age of concrete in minutes and track time in AEST or select time zone. The concrete sensor devices are in a waterproof housing can have in-built plastic “straps” for ease of tying them into the cast concrete. The concrete sensor devices can be installed to a depth below the surface of 0 mm and 150 mm within concrete and still achieve signal penetration. The concrete sensor devices may be specifically designed square plastic housings to assist in ease of installation. The concrete sensor devices will be able to be toggled on and off and also paired by using a magnetic switch at the top of the sensor. The concrete sensor device can be turned off from a central gateway for years, than if the structure is being refurbished or sold than the sensors are turned back on at a future date to begin transmitting data again.

The concrete sensor devices may be operated under the following usage scenarios. The curing mode (typically active from 0 to 60 days of concrete pouring) is a high powered, high data transfer environment, providing users and stakeholders with near real-time data information through the critical curing process. Every 30 minutes temperature and humidity data will be sent. In the structure mode (typically active from 60 days of concrete pouring), the concrete sensor device switches to a low powered and low data transfer mode and the accelerometer is activated. The accelerometer will provide any tilt or loading change alerts on a daily or as happening basis. The concrete sensor device will also provide a daily update of temperature and humidity. In the structure mode, power consumption may be further reduced by storing data and then transmitting it in a burst mode at protracted intervals, perhaps weekly or longer. The concrete sensor device can also perform on-site tilt analysis to generate immediate alerts for any significant changes in pose.

A brief example of a method of operation of the system including the concrete sensor devices will now be outlined.

On day 1, the concrete sensor device is installed and concrete poured. The concrete sensor device starts transmitting near real-time information of temperature and humidity. This data is then displayed through the web portal, and users can activate alert emails or SMS to be sent at certain milestones of curing (e.g., when 10 MPa, 22 MPa, 40 MPa of estimated strength is achieved and similar when certain percentages of humidity are reached etc.). Throughout days 2-60 after the concrete has been poured, the concrete sensor device continues to act as per day 1. Then, after day 60, the concrete sensor device switches to low powered and low data transfer mode and accelerometer is activated. This will record any change in tilt (deflection) and load stiffness which will identify any possible load path change, failure or monitoring of deflection/tilt in the concrete element. This data will also be displayed through the web-portal. Users can activate alert emails or SMS to be sent if accelerometer records data outside a certain tolerance, Also a “failure” tolerance will be benchmarked if surpassed it sends high priority alert emails and notifications to users to notify of major departure from specification and structure to be immediately checked. It will also continue to give once daily updates of temperature and humidity.

The concrete sensor devices may utilise long life Lithium Thionyl Chloride batteries, which can remain operational for up to 40 years. In sleep mode, the concrete sensor device may draw approximately 20 micro amps and its on-board Real-Time Clock (RTC) has the ability to wake itself at any selected date/time or interval. Burst power for radio transmission is provided by an on-board Electrostatic Double Layer “super” Capacitor, (EDLC) which is trickle charged from the battery.

The concrete sensor device can be stored for decades on the shelf until activated by an external magnet brought into close proximity (approximately 10 mm) to an on-board magnetic relay. The concrete sensor device can also then be deactivated by placing the magnet for a minimum of 6 seconds. An on-board ultra low leakage current activation circuit is used to facilitate this. Additional concrete sensor devices can also be installed in the concrete and put to sleep to only be activated if long term access is desired.

In summary, the concrete sensor device and systems using these devices can allow critical project data to be viewed without local presence. The concrete sensor devices can allow stakeholders to remotely view and monitor their project from anywhere in the world. The concrete sensor device can allow works to progress safely knowing what strength and characteristics the concrete is. The concrete sensor device can also allow works to progress immediately without having to wait for conventional methods of lab testing, and without requiring anyone to be on site to gain the data.

The concrete sensor device can further allow for the project's concrete data to be stored and collated for future use. Future clients can access the concrete sensor devices in real-time years after the construction is finished, so engineers can investigate for refurbishment of the building knowing its exact state and condition. Accordingly, the use of the concrete sensor devices and associated systems can add significant value to concrete construction projects.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

Although the description above mostly relates to application to a concrete structure, the structure sensor device(s) can also be embedded in ground, rock, soil or earth structures for monitoring temperature, humidity, tilt, movement and vibration of earth and rock structures. Such structure for example can include a tailings dam.

We claim:

1. A structure sensor device for use in monitoring physical properties within a body of concrete, rock or soil structure, the structure sensor device being configured to be embedded inside the body of structure in use, the structure sensor device comprising:
a. a sealed housing;
b. a plurality of sensors for measuring physical properties within the body of structure, comprising:
i. a temperature sensor; and
ii. a moisture sensor;
c. a wireless transceiver for wirelessly communicating with a gateway device located outside of the body of structure, the wireless transceiver being configured for wirelessly communicating at frequencies of less than 1 GHz; and
d. a controller configured to:
i. obtain sensor data from the sensors;
ii. generate monitoring data using at least some of the sensor data; and
iii. cause the monitoring data to be transmitted to the gateway device using the wireless transceiver, at predetermined intervals.

2. The structure sensor device according to claim 1, wherein the plurality of sensors further comprises an accelerometer.

3. The structure sensor device according to claim 2, wherein the controller is configured to use sensor data obtained from the accelerometer to determine at least one of:
a. a tilt angle of the structure sensor device;
b. stiffness of the structure; and
c. vibration in the structure.

4. The structure sensor device according to claim 3, wherein the structure sensor device is configured to operate under different operating modes, the controller being configured to:
a. generate the monitoring data in accordance with a current operating mode; and
b. cause the monitoring data to be transmitted to the gateway device at intervals selected in accordance with the current operating mode.

5. The structure sensor device according to claim 4, wherein the structure is concrete, and wherein the operating modes are comprised of:
a. a curing mode for use during curing of the concrete; and
b. a structure mode for use after curing of the concrete.

6. The structure sensor device according to claim 5, wherein the controller is configured to significantly reduce power consumption of the structure sensor device in the structure mode, compared to the curing mode.

7. The structure sensor device according to claim 6, wherein the controller is configured to cause the monitoring data to be transmitted at shorter intervals in the curing mode and longer intervals in the structure mode.

8. The structure sensor device according to claim 5, wherein the controller is configured to, in the curing mode, generate the monitoring data using sensor data obtained from the temperature sensor and the moisture sensor.

9. The structure sensor device according to claim 5, wherein the plurality of sensors further comprise an accelerometer, and wherein the controller is configured to, in the structure mode, generate the monitoring data using sensor data obtained from at least the accelerometer.

10. The structure sensor device according to claim 9, wherein the controller is configured to, in the structure mode, generate the monitoring data additionally using sensor data obtained from the temperature sensor and the moisture sensor.

11. The structure sensor device according to claim 10, wherein the controller is configured to, in the structure mode:
a. determine whether an alert condition exists based on sensor data obtained from the accelerometer;
b. in the event of determining that an alert condition exists, generating alert data; and
c. causing the alert data to be transmitted to the gateway device using the wireless transceiver.

12. The structure sensor device according to claim 5, wherein the controller is configured to initially operate under the curing mode and switch to the structure mode after a predetermined period of time.

13. The structure sensor device according to claim 1, wherein the wireless transceiver is configured for wirelessly communicating with the gateway device using a LoRa wireless communication protocol.

14. The structure sensor device according to claim 1, wherein the wireless transceiver is configured for wirelessly communicating with the gateway device in a frequency range of at least one of:
a. between 433 MHz and 928 MHz;
b. between 902 MHz and 928 MHz; and
c. between 915 MHz and 928 MHz.

15. The structure sensor device according to claim 1, wherein the wireless transceiver is configured for wirelessly communicating with another structure sensor device.

16. The structure sensor device according to claim 15, wherein the controller is configured to cause the monitoring data to be transmitted to the gateway device via the other structure sensor device.

17. The structure sensor device according to claim 1, wherein the moisture sensor is configured to measure a relative humidity within the structure.

18. The structure sensor device according to claim 1, further comprising: two probes, the controller being configured to measure electrical signals in the two probes and determine a distance between the two probes based on the measured electrical signals, to thereby allow shrinkage of the structure to be monitored.

19. The structure sensor device according to claim 1, further comprising: a magnetic switch that can be activated by bringing an external magnet into proximity of the magnetic switch, and the structure sensor device is configured to respond to activation of the magnetic switch by at least one of:
a. switching on the structure sensor device;
b. switching off the structure sensor device;
c. switching between operation modes; and
d. causing the structure sensor device to be wirelessly paired with a gateway device.

20. The structure sensor device according to claim 1, wherein the structure sensor device is configured to be remotely switched off using the gateway device.

21. The structure sensor device according to claim 1, wherein, the controller is configured to conserve power by alternating between a sleep state and an awake state at predetermined intervals, and wherein at least the wireless transceiver is deactivated in the sleep state.

22. The structure sensor device according to claim 1, further comprising: a power supply being comprised of a battery and a super capacitor.

23. The structure sensor device according to claim 22, wherein the battery is used to provide a steady low operating current and the super capacitor is used to provide high short duration pulse currents for powering the wireless transceiver transmitting the monitoring data at the predetermined intervals.

24. The structure sensor device according to claim 1, wherein the housing does not include any:
a. external control inputs;
b. external electrical connections; and
c. external antennae.

25. The structure device according to claim 1, wherein the housing comprises at least one of:
a. fastening points for attachment of separate fastening devices;
b. holes for attachment of cable ties; and
c. integral fastening devices.

26. A system for monitoring physical properties within one or more bodies of structure, the system including:
a. a plurality of structure sensor devices according to claim 1, each structure sensor device being embedded inside a body of structure in use; and
b. a gateway device located proximate to one or more bodies of structure in use, the gateway device comprising:
i. a gateway wireless transceiver for wirelessly communicating with the plurality of structure sensor devices, the gateway wireless transceiver being configured for wirelessly communicating at frequencies of less than 1 GHz;
ii. a gateway wireless network interface for wirelessly communicating with a processing system via a wireless communications network; and
iii. a gateway processing system configured to:
1. receive monitoring data from the plurality of structure sensor devices; and
2. transmit at least some of the monitoring data to the processing system via the wireless communications network.

27. The system according to claim 26, wherein the gateway wireless network interface comprises a wireless modem for wirelessly communicating with a server processing system via the Internet.

28. The system according to claim 27, wherein the server processing system is configured to allow a remote user device to access monitoring data from the server processing system via the Internet.

29. The system according to claim 28, wherein the server processing system is configured to allow the remote user device to access the monitoring data using one of a web portal and an application programming interface.

30. The system according to claim 29, wherein:
a. the gateway processing system is configured to:
i. receive alert data from one of the plurality of structure sensor devices; and
ii. relay the alert data to the server processing system; and
b. the server processing system is configured to:
i. upon receipt of the alert data, generate an alert notification; and
ii. transmit the alert notification to the remote user device.

31. The system according to claim 30, wherein:
a. the server processing system is configured to:
i. receive, from the remote user device, a user command for controlling the operation of a selected one or more of the plurality of structure sensor devices; and
ii. transmit the user command to the gateway device; and
b. the gateway processing system is configured to transmit structure sensor device control signals to the selected one or more of the plurality of structure sensor devices.

32. The system according to claim 31, wherein the server processing system is configured to determine an estimated strength of the concrete based on received monitoring data.

33. The system according to claim 32, wherein the server processing system is configured to:
a. determine whether the estimated strength of the structure has reached a predetermined threshold; and
b. in the event of a successful determination, generate a strength notification; and
c. transmit the strength notification to the remote user device.

34. The system according to claim 33, wherein the gateway wireless network interface is comprised of a wireless access point for allowing a local user device to wirelessly connect to the gateway device to allow the local user device to access monitoring data directly from the gateway device.
